(19) United States
(12) Patent Application Publication
(10) Pub. No.: US 2020/0115708 A1
(43) Pub. Date: Apr. 16, 2020

(54) DNA probe library for hybridization with microsatellite instability related microsatellite loci, detection method and kit

(71) Applicant: GENESEEQ TECHNOLOGY INC., Nanjing (CN)

(72) Inventors: Yang SHAO, Nanjing (CN); Siming JIANG, Nanjing (CN); Xiaonan WANG, Nanjing (CN); Xue WU, Nanjing (CN); Zhili CHANG, Nanjing (CN); Xiangyuan MA, Nanjing (CN); Xian ZHANG, Nanjing (CN)

(73) Assignee: GENESEEQ TECHNOLOGY INC., Nanjing (CN)

(21) Appl. No.: 16/621,234

(22) PCT Filed: Jun. 6, 2018
(86) PCT No.: PCT/CN2018/090084

§ 371 Date: Dec. 10, 2019

(30) Foreign Application Priority Data
Aug. 1, 2017 (CN) 201710647677.8

(51) Int. Cl. C12N 15/10; C12Q 1/6874

(52) U.S. Cl. CPC C12N 15/1093; C12Q 1/6874

(57) ABSTRACT

A DNA probe library for hybridization with microsatellite loci associated with microsatellite instability (MSI) detection. The said DNA probe library comprises one or more DNA probes that are capable of hybridizing with the MSI status-related microsatellite loci. Among the said DNA probes, probes for the MSI-related microsatellite loci are shown in the following sequences: SEQ ID NOS. 1-66. In addition, the present invention provides a method for enriching and detecting the MSI-related microsatellite loci using the probe library. The combination of this method and next-generation sequencing technology (NGS) can greatly improve the sensitivity, accuracy and comprehensiveness of the MSI detection.

### CROSS REFERENCE TO RELATED APPLICATIONS

This application is the national phase entry of International Application No. PCT/CN2018/090084, filed on Jun. 6, 2018, which is based upon and claims priority to Chinese Patent Application No. 201710647677.8, filed on Aug. 1, 2017, the entire contents of which are incorporated herein by reference.

### TECHNICAL FIELD

The present invention relates to the field of gene detection, and in particular to a method for enrichment and detection of microsatellite instability (MSI) related microsatellite loci. The said method can accurately enrich specific fragments of the MSI-related microsatellite loci. The resulting DNA sample library can be further combined with the next-generation sequencing technology (NGS) to quantitatively assess a patient's MSI status by bioinformatics analysis, providing guidance and theoretical basis for diagnosis, prognosis and design of clinical treatment plans of tumor.

### BACKGROUND

Microsatellite instability (MSI) refers to the change in the length of a microsatellite allele due to abnormal insertion or deletion in repeats during DNA replication, and the change cannot be corrected by the DNA mismatch repair system (MMR) due to various reasons (e.g. suppression of gene expression by promoter methylation, or inactivation and truncation mutations occurring in the genes for the DNA mismatch repair mechanism). MSI is specifically characterized by the fact that the length of the microsatellite loci with a relatively stable number of repeat units in normal tissues becomes unstable in abnormal tissues and shows changes.

Since Aaltonen et al. found in 1993 that hereditary nonpolyposis colorectal cancer (HNPCC), also known as Lynch syndrome, has high frequency of MSI in cells, the researchers have successively found the existence of MSI in a series of cancers such as lung cancer, gastric cancer and endometrial cancer. MSI has a high prevalence in certain cancers. For example, approximately 15% of colorectal cancers are MSI tumors. In particular, the incidence of MSI in early-onset colorectal cancer reaches 30%, while the proportion of MSI tumors in HNPCC is as high as 90%. Clinical studies have shown that state II/III intestinal cancer patients with high-frequency MSI (MSI-H) have a better prognosis and cannot benefit from adjuvant chemotherapy with fluorouracils (e.g. 5-FU). Therefore, the MSI detection has multiple clinical pathological significance.

Regarding the use of the MSI detection in the treatment of colorectal cancer, the National Cancer Institute (NCI) issued the Bethesda Guidelines in 1997. The Bethesda Guidelines recommend five microsatellite loci (BAT-26, BAT-25, D2S123, D5S346, and D17S250) that can be used for the detection of MSI in colorectal cancer. In addition, the Bethesda Guidelines classified the MSI and defined each category, namely:

1. High frequency MSI (MSI-H): Two or more of the recommended loci show changes in length of the repeats;

2. Low frequency MSI (MSI-L): One of the recommended loci shows changes in length of the repeats;

3. Microsatellite stability (MSS): There is no change in the length of the repeats in the recommended loci.

Among the initial five recommended loci proposed by Bethesda Guidelines, the stability of three loci with dinucleotide repeats (D2S123, D5S346 and D17S250) was controversial at the 2002 NCI meeting: Suraweera's research team pointed out that for patients with MSI-H, the detection sensitivity can be improved when the above three loci with dinucleotide repeats are replaced by the following three loci with mononucleotide repeats, NR21, NR22 and NR24. In 2004, Bacher's research group showed that the detection sensitivity using the loci with mononucleotide repeats was 92%-100%, and the specificity for MSI-H cases was as high as 99.5%-100%. This conclusion was further confirmed by the Rosa M. Xicola's group in 2007. In addition to the Bethesda Guidelines, Promega Biotech Co., Ltd. developed its own MSI analysis system, which uses the locus with mononucleotide repeats, Mono27, to replace the NR22 proposed by Suraweera et al., and also adds two loci with pentanucleotide repeats, Penta C and Penta D, that are highly diverse in the human population, for sample quality control.

Because of its high sensitivity and specificity, MSI detection was officially listed as the primary detecting item in the National Comprehensive Cancer Network Guidelines for Colorectal Cancer (CRC) Screening in 2011, which stated that the following populations should receive the MSI detection:
Patients under 50 years of age who are diagnosed with colorectal cancer;
Patients with synchronous or metachronous HNPCC-like tumors, regardless of age;
Patients who have one or more first-degree relatives diagnosed with HNPCC tumors, and at least one of these relatives are less than 50 years old;
Patients who have two or more first-degree or second-degree relatives diagnosed with HNPCC-like tumors, regardless of age.

Recent studies have shown that MSI detection also plays an important guiding role in tumor immunotherapy. A number of studies have shown that colon cancer patients with MSI-H display better prognosis after receiving PD-1 antibody therapy compared to patients with MSI-L or MSS. This has also been verified in other cancer types. In May 2017, the US FDA granted accelerated approval of the immunotherapeutic drug Perbrolizumab (Keytruda) for solid-tumor patients with MSI-H or DNA mismatch repair defects, who have inoperable or advanced metastases and have progressed after previous treatment. This is the first FDA-approved therapy that is tumor type-agnostic, herein supporting that MSI detection has an extensive and valuable role in clinical guidance.

The existing MSI detection mainly uses the following two technologies:

1. PCR detection (MSI-PCR): with use of specific primers, the microsatellite locus is amplified by PCR or multiplex fluorescent PCR. The amplified product is subjected to gel electrophoresis or Sanger fragment size analysis to determine whether its product fragment has a change in the migration compared with the normal control, thereby determining the MSI status.

2. DNA mismatch repair defect detection: it directly detects gene mutations in related genes responsible for MSI phenomenon, mainly the DNA mismatch repair system (MMR) genes, or detects the level of proteins expressed by these genes via immunohistochemistry.

In these two technologies, the PCR detection is currently the most popular, and is also recognized as the most cost-effective detection method. However, the regular PCR method has the shortcomings including cumbersome operation procedure, time-consuming, low sensitivity, and high uncertainty in detection results. In the multiplex PCR detection, the interaction between different primers is very complicated, potentially causing higher level of unspecific/off-target amplifications. Therefore, there are high requirements for the selectivity and concentration of primers, which undoubtedly greatly increases the cost of detection. For the detection of DNA mismatch repair gene defects, the traditional gene sequencing methods, such as Sanger sequencing, also have limitations such as high cost, low throughput, and low precision. The immunohistochemistry method has low specificity and reproducibility, but has a high requirement on the sample quality as well as complicated procedures. Finally, due to the limited number of microsatellite loci that can be detected by existing conventional MSI detection methods, it is difficult to distinguish between the high frequency MSI-H and the low frequency MSI-L. Therefore, there is an urgent need to develop a novel MSI detection method which can detect more microsatellite loci simultaneously and is simpler, faster, more sensitive and higher reproducible as compared to the existing methods, to meet the clinic needs.

### SUMMARY

The next-generation sequencing technology (NGS) has a great application potential in MSI detection. The present invention provides a complete scheme of the NGS-based MSI status detection, which can greatly simplify the detection process and reduce the cost of the detection. With a large number of literature searches and experimental verifications, the inventors have identified 22 optimized microsatellite loci suitable for MSI status assessment, and developed a method for capturing MSI-related microsatellite loci based on hybridization and selection, which can be used for targeted enrichment of fragments of the MSI-related microsatellite loci. The fragments of the microsatellite loci enriched by the described method can be selectively applied to various technologies for genetic detecting, in particular to the NGS-based MSI detection.

### The First Aspect of the Invention

The present invention identifies twenty-two (22) microsatellite loci with mononucleotide repeats in human genome for MSI status detection (see Table 1). These loci are characterized that the number of their repeat units is relatively fixed in normal cells, and their stability is verified in a population of more than 2000 Chinese people; while in the MSI status, the number of repeat units in these loci is polymorphic.

TABLE 1
Information about microsatellite loci

| Locus | Microsatellite | Repeat | Genomic location |
|---|---|---|---|
| number | locus name | (number) | (Human Hg19) |
| MS-1 | BAT25 | T(25) | chr4: 55,598,212-55,598,236 |
| MS-2 | BAT26 | A(27) | chr2: 47,641,560-47,641,586 |
| MS-3 | NR24 | T(23) | chr2: 95,849,362-95,849,384 |
| MS-4 | NR21 | T(21) | chr14: 23,652,347-23,652,367 |
| MS-5 | Mono27 | A(27) | chr2: 39,536,690-39,536,716 |
| MS-6 | NR22 | T(21) | chr11: 125,490,766-125,490,786 |
| MS-7 | NR27 | A(26) | chr11: 102,193,509-102,193,534 |
| MS-8 | BAT40 | T(37) | chr1: 120,053,341-120,053,377 |
| MS-9 | CUL-22 | A(22) | chr2: 225,422,601-225,422,622 |
| MS-10 | MET-15 | T(15) | chr7: 116,409,676-116,409,690 |
| MS-11 | ATM-15 | T(15) | chr11: 108,114,662-108,114,676 |
| MS-12 | RB1-13 | T(13) | chr13: 48,954,160-48,954,172 |
| MS-13 | NF1-26 | T(26) | chr17: 29,559,062-29,559,087 |
| MS-14 | DDR-11 | A(11) | chr1: 162,736,822-162,736,832 |
| MS-15 | FANC-21 | A(21) | chr3: 10,076,009-10,076,029 |
| MS-16 | MITF-14 | T(14) | chr3: 69,988,438-69,988,451 |
| MS-17 | PKHD-18 | A(18) | chr6: 51,503,598-51,503,615 |
| MS-18 | PTK-16 | A(16) | chr8: 141,754,889-141,754,904 |
| MS-19 | RET-14 | T(14) | chr10: 43,595,837-43,595,850 |
| MS-20 | CBL-17 | T(17) | chr11: 119,144,792-119,144,808 |
| MS-21 | PTPN-17 | T(17) | chr12: 112,893,676-112,893,692 |
| MS-22 | SMAD-18 | A(18) | chr18: 45,395,846-45,395,863 |

### The Second Aspect of this Invention

The present invention provides a DNA probe library for hybridization with microsatellite instability (MSI)-related microsatellite loci, which includes a DNA probe library capable of hybridizing to the twenty-two (22) microsatellite loci with mononucleotide repeats in the genomic region.

The method for designing the probe library is:

A first probe and a second probe are individually designed for each of the microsatellite loci. One end of the first probe specifically binds upstream of sequence of the microsatellite locus and the other end specifically binds to the internal region of the microsatellite locus. One end of the second probe specifically binds to the internal region of the microsatellite locus and the other end specifically binds to a downstream region of the sequence of the microsatellite locus. The third probe has two ends specifically binding to the upstream and downstream regions of the microsatellite locus respectively.

The probes in the probe library have a length of 80 to 120 bp, more preferably 120 bp.

The DNA probe library includes any one of the probes with nucleotide sequences as shown in SEQ ID NOS. 1 to 66, or a probe having the same function thereof.

Preferably, the probe library includes all of the probes mentioned above.

Preferably, the probe having the same function thereof refers to probe with a substitution and/or deletion and/or addition of one or more nucleotides in any one of the probes shown in SEQ ID NOs. 1-66 and having the same hybridizing and capturing function.

Preferably, the probe having the same function thereof has ≥80% identical bases, more preferably ≥90% identical bases, and most preferably ≥95% identical bases to the original probe.

### The Third Aspect of the Invention

The present invention provides a method for enriching fragments of MSI-related microsatellite loci, comprising the steps of:

1) obtaining a DNA sample library from a subject;

2) obtaining a DNA probe library capable of hybridizing with the MSI-related microsatellite loci;

3) hybridizing the DNA probe library to the DNA sample library; and

4) isolating the hybridization product of step 3), followed by release of the hybridization-enriched fragments of the MSI-related microsatellite loci.

The DNA sample library in the step 1) consists of double-stranded DNA fragments and the step 1) includes extracting whole genome DNA and then fragmenting it.

The subject is a mammal, preferably a human, and the whole genomic DNA is extracted from cell, tissue or body fluid samples of the subject.

Preferably, the DNA fragments are 150-600 bp in length.

More preferably, the DNA fragments are 200 bp or 350 bp in length.

The DNA probe library in the step 2) is the DNA probe library mentioned above. In particular, the DNA probe library includes one or more of the DNA probes capable of hybridizing to the fragments of the MSI-related microsatellite loci. These DNA probes' sequences are shown in SEQ ID NOS. 1-66.

In addition, the step 3) comprises:

3-1) labeling the DNA probes in the DNA probe library with selectable markers; and

3-2) hybridizing the DNA probe library with the DNA sample library.

Preferably, the selectable markers in the step 3-1) are biotin; further preferably, the step 3-2) includes incubating the DNA probe library with the DNA sample library for 24 hours at 65° C. in a PCR thermocycler.

Therefore, in the step 4) of the method, the hybridization product is preferably isolated using the selectable markers on the DNA probes. Further preferably, the selectable markers in the step 3-1) are biotin, and in the step 4) the hybridization product is isolated by affinity of streptavidin-biotin.

### The Forth Aspect of the Invention

The present invention also provides a method for detecting change in number of repeat units in MSI-related microsatellite loci, comprising:

1) enriching fragments of the MSI-related microsatellite loci according to the above method; and

2) detecting the change in the number of repeat units in the MSI-related microsatellite loci.

Preferably, in the step 2), using next-generation sequencing technology (NGS), the enriched fragments of the MSI-related microsatellite loci are sequenced to detect the change in the number of repeat units in the MSI-related microsatellite loci.

### The Fifth Aspect of the Invention

The present invention provides a kit for enriching fragments of MSI-related microsatellite loci, comprising the DNA probe library mentioned above.

### The Sixth Aspect of the Invention

The kit is used for microsatellite instability (MSI) related microsatellite loci detection for non-therapeutic and non-diagnostic purposes.

### Beneficial Effect

In summary, the inventors have developed a method for capturing specific MSI-related microsatellite loci based on hybridization and selection, by which tens of thousands of enriched fragments of the MSI-related microsatellite loci can be obtained. The samples of the enriched fragments of the MSI-related microsatellite loci can be selectively applied to various technologies for genetic detection, especially the next-generation sequencing technology which can be used for detecting for example gene mutation, deletion, addition, and transversion to achieve efficient and accurate results, providing valuable theoretical and clinical guidance for the follow-up treatment of related symptoms.

Moreover, the fragments of the MSI-related microsatellite loci enriched by the method of the present invention can be used for structural mutation detection based on the next-generation sequencing technology. This application has the following beneficial effects:

The present invention provides a gene enrichment method and a specific DNA probe library obtained by screening, which can enrich the MSI-related microsatellite loci by tens of thousands of folds, so that various mutations in the repeats of the MSI-related microsatellite loci can be accurately detected by sequencing the MSI-related microsatellite loci via the next-generation sequencing technology. Moreover, by combining with the next-generation sequencing technology, simultaneous detection on multiple types of gene mutations at multiple loci can be achieved; the detection accuracy is high according to the present invention. Conventional techniques such as gene microarray technology usually need to be repeated more than twice to determine the detection results, while the present invention allows repeated sequencing of a single base in a single reaction, which ensures data accuracy and shortens the detection period; the detection sensitivity is high according to the present invention. The data generated by the present invention can achieve a resolution to the base-level compared with those from conventional detection techniques, greatly improving the sensitivity.

### BRIEF DESCRIPTION OF THE DRAWINGS

FIG. 1: Exemplary process flow chart of the technical solution of the present invention, wherein the target DNA fragment is enriched and used for detection of gene structural mutations based on the next-generation sequencing technology.

FIG. 2: A schematic diagram of design strategy for probes provided by the present invention.

FIG. 3: A PCR polymorphism diagram for a representative MSI-H sample.

FIG. 4-9: Sequencing imagines on different MSI sensitive loci from MSI-H patients.

FIG. 10: A PCR polymorphism diagram for a representative MSS sample.

FIG. 11-16: Sequencing imagines on different MSI sensitive loci from MSI-H samples.

### DETAILED DESCRIPTION OF THE EMBODIMENTS

The invention will now be further described in detail by way of specific embodiments. However, it will be understood that the following examples are merely illustrative of the invention and should not be construed as limiting the scope of the invention. While specific techniques or conditions are not indicated in the examples, they are carried out according to the techniques or conditions described in the literature of the field or in accordance with the product manuals. The used reagents or instruments which are not provided with the manufacturer information are conventional and commercially-available products.

The term “DNA” as used herein is deoxyribonucleic acid (abbreviated as DNA) which is a double-stranded molecule composed of deoxyribonucleotides. It can form genetic instructions to guide biological development and vital function. Its base sequence constitutes genetic information, therefore playing an important role in the diagnosis of genetic diseases.

The term “next-generation sequencing technology” as used herein refers to a second generation high throughput sequencing technology and a higher throughput sequencing method developed later. Next-generation sequencing platforms include, but are not limited to, Illumina (Miseq, Hiseq2000, Hiseq2500, Hiseq3000, Hiseq4000, HiseqX Ten, etc.), ABI-Solid, and Roche-454 sequencing platforms. As the sequencing technology continues to evolve, it can be appreciated that other sequencing methods or apparatus can also be used for this detection. According to a specific example of the present invention, the nucleic acid tag according to an embodiment of the present invention can be used for at least one of the Illumina, ABI-Solid, and Roche-454 sequencing platforms. Next-generation sequencing technologies, such as Illumina sequencing technology, have the following advantages: (1) High sensitivity: Next-generation sequencing, such as Miseq has high sequencing flux which can generate up to 15G base data in one experimental process. In the case where the number of sequences is constant, the high data throughput allows for higher sequencing depth for each sequence so that the lower levels of mutations can be detected. In the meanwhile, because of the high sequencing depth, the mutation sites can have high coverage leading to a more reliable result. (2) High throughput and low cost: With the tag sequence according to an embodiment of the present invention, tens of thousands of samples can be detected by a single sequencing process, thereby greatly reducing the cost.

The “mutation”, “nucleic acid variation”, and “gene variation” in the present invention are used interchangeably. The “SNP” (SNV), “CNV”, “insert deletion” (indel), and “structural variation” (SV) in the present invention are defined as usual, but the size of each variation is not particularly limited in the present invention, therefore crossovers among the several variations, such as when the insertion/deletion is a large fragment or even a whole chromosome, belonging to the copy number variation (CNV) or chromosomal aneuploidy also belongs to SV These types of crossover variations do not prevent the methods and/or device of the present invention being performed to obtain the results described.

The present invention provides a method for enriching MSI-related microsatellite loci. Specifically, the method of the present invention comprises: extracting genomic DNA from a cell, body fluid or tissue sample of a mammal such as a human, processing to obtain fragmented double-stranded DNA as a DNA sample library; and further, designing DNA probes hybridized with the MSI-related microsatellite locus being enriched, and selecting a plurality of probes to form a DNA probe library; then, the DNA sample library is hybridized with the DNA probe library, thereby the fragments of MSI-related microsatellite loci were enriched in the DNA sample library. According to a specific embodiment of the present invention, each probe in the DNA probe library can be biotinylated first, and then the hybridization product is bound by streptavidin magnetic beads after hybridization, and then released from the magnetic beads becoming the enriched fragments of MSI-related microsatellite loci. After adaptive treatment, the next-generation gene sequencing can be used to detect and confirm the structural mutation of the MSI-related microsatellite loci.

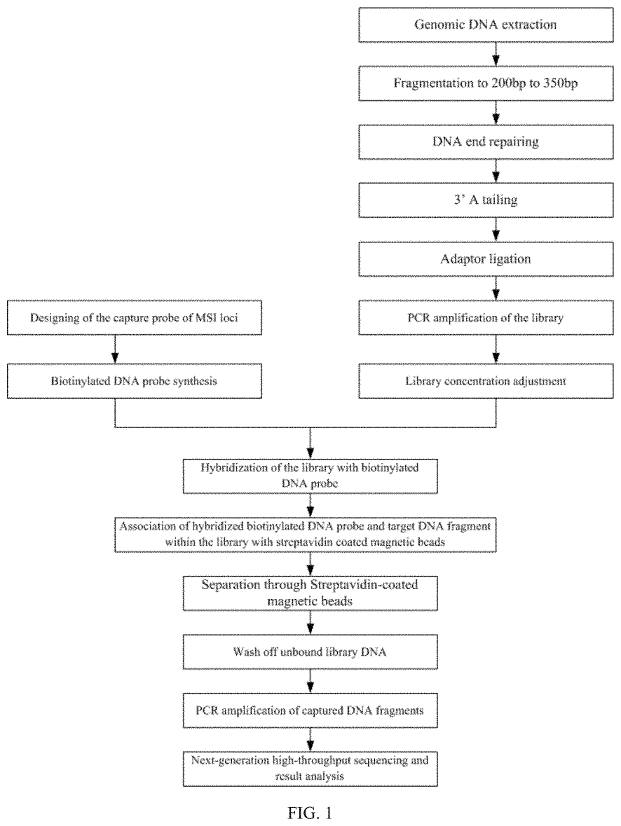

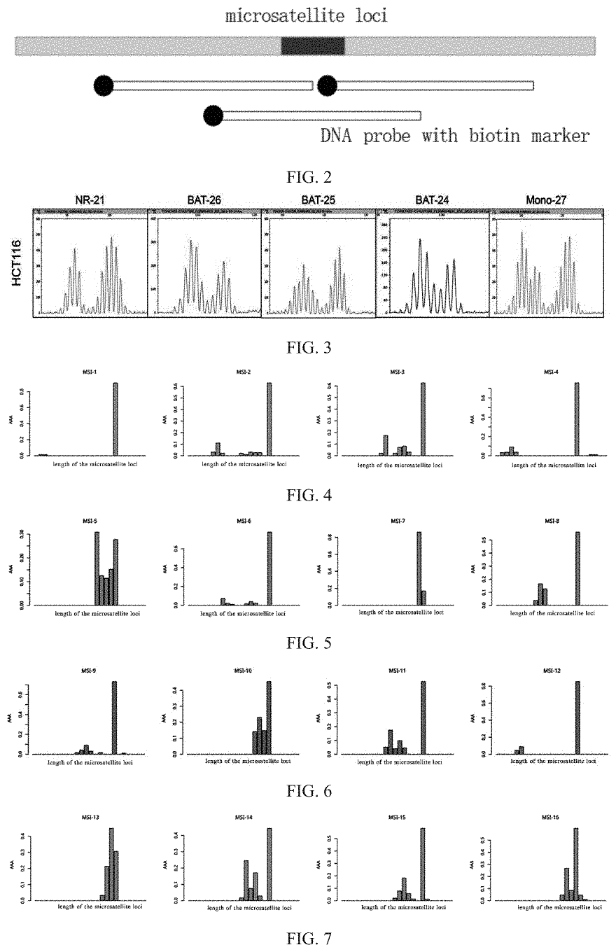

The present invention is exemplarily illustrated by using the enriched fragments of MSI-related microsatellite loci to detect the gene structural mutations based on the next-generation sequencing technology, wherein the overall process flow is shown in FIG. 1.

### I. Prepare the DNA Sample Library

1. Prepare a Genomic DNA Sample (the DNA Sample Library Obtained in this Way is Called a “Genome-Derived DNA Sample Library”)

1.1 DNA Extraction

DNA extraction, including fresh tissue, fresh blood and cells, formalin fixed and paraffin embedded samples, and commercial company extraction kits, is operated according to the instructions in the manual.

DNA template quality and concentration are measured using a spectrophotometer and a gel electrophoresis system. It is considered as qualified when the absorbance of the dsDNA template at 260 nm is greater than 0.05, and the ratio of absorbance A260/A280 is between 1.8 and 2.

1.2 DNA Fragmentation

Three (3) μg of high quality genomic DNA is diluted to 120 ml with low TE buffer. The DNA is fragmented according to the instruction of the tissue homogenizer, and the fragment length is 150-600 bp, preferably 200 bp or 350 bp.

DNA is purified by column provided in the commercial purification kit.

1.3 DNA Sample Library Quality Detecting

Qualitative and quantitative analysis of DNA using a bioanalyzer confirm that the peak length of the DNA fragment is reasonable.

2. DNA End Repairing

End-repairing of a DNA fragment can be carried out using a Klenow fragment, T4 DNA polymerase, and T4 polynucleotide kinase, wherein the Klenow fragment has 5′-3″ polymerase activity and 3′-5′ polymerase activity, but lacks 5′-3′ exonuclease activity. Thereby, the DNA fragment can be easily and accurately repaired at the end. According to an embodiment of the present invention, the step of purifying the end-repaired DNA fragment may further be included, whereby subsequent processing can be conveniently performed.

Using T4 polymerase and Klenow E. coli polymerase fragments, the DNA 5′ overhang sticky ends are filled and the 3′ overhang sticky ends are flattened to produce blunt ends for subsequent blunt-end ligation. The reaction is carried out in the PCR thermocycler at 20° C. for 30 minutes.

TABLE 2
DNA end repairing reaction composition

| Reaction material | Volume |
|---|---|
| Purified DNA sample library | 50 μl |
| Phosphorylation buffer | 10 μl |
| Deoxybase mixture dNTP (10 mM each) | 4 μl |
| T4 DNA polymerase | 5 μl |
| Klenow E. coli polymerase fragments | 1 μl |
| T4 polynucleotide kinase | 5 μl |
| Nuclease-free water | Add to make the total |
| | volume as 100 μl |

DNA is purified by column provided in the commercial company purification kit.

4. Add Base A to the 3′ End of the DNA Sample.

A base A is added to the 3′ end of the end-repaired DNA fragment to obtain a DNA fragment with a sticky end A. According to one embodiment of the present invention, Klenow (3′-5′exo-), Klenow having 3′-5′ exonuclease activity, can be used to add base A at the 3′ end of the end-repaired DNA fragment. Thereby, the base A can be easily and accurately added to the 3′ end of the DNA fragment which has been repaired at the end. According to an embodiment of the present invention, the step of purifying the DNA fragment with the sticky end A may further be included, whereby subsequent processing can be conveniently performed.

The reaction is carried out in the PCR thermocyclerthermocycler at 37° C. for 30 minutes.

TABLE 3
Add Base A end reaction composition

| Reaction material | Volume |
|---|---|
| DNA sample library | ~30 μl |
| 10X Klenow E. coli polymerase | 5 μl |
| buffer | |
| Deoxy base dATP (1 mM) | 10 μl |
| Klenow E. coli polymerase fragments | 3 μl |
| Nuclease-free water | Add to make the total |
| | volume as 50 μl |

DNA is purified by column provided in the commercial company purification kit.

5. Add Adapters at Both Ends of the DNA

TABLE 4
Reaction composition for adding adapters at the DNA ends

| Reaction material | Volume |
|---|---|
| DNA sample library | ~15 μl |
| 2X T4 DNA polymerase buffer | 5 μl |
| DNA adapters | 6 μl |
| T4 DNA polymerase | 3 μl |
| Nuclease-free water | Add to make the total |
| | volume as 50 μl |

DNA is purified by column provided in the commercial company purification kit.

7. Amplification of the DNA Template

Polymerase chain reaction (PCR) is performed in the PCR thermocycler.

TABLE 5
PCR reaction composition

| Reaction material | Volume |
|---|---|
| DNA sample library with adapters added | ~30 μl |
| 10X High-accuracy ultra-fidelity DNA | 5 μl |
| polymerase buffer | |
| High-accuracy ultra-fidelity DNA polymerase | 1 μl |
| Adapter forward primer | 1 μl |
| Adapter reverse primer | 1 μl |
| Nuclease-free water | Add to make the total |
| | volume as 50 μl |

PCR conditions: placed in the PCR thermocycler, pre-denatured at 98° C. for 30 seconds, denatured at 98° C. for 30 seconds, annealed at 65° C. for 30 seconds, extended at 72° C. for 30 seconds, repeated 4-6 times. Finally, it is extended at 72° C. for 5 minutes.

PCR amplification product is purified by column provided in the commercial company purification kit.

9. Quality Analysis of the DNA Sample Library after Amplification

DNA biochemical quantitative analysis is performed using a bioanalyzer, which confirms that the peak length of the fragment after purification is reasonable, about 200 bp.

For the obtained DNA sample library, if the DNA concentration is less than 150 ng/μl, the sample must be dried at a low temperature (lower than 45° C.) by a vacuum concentrator, and then dissolved in the nuclease-free water to the desired concentration.

### II. Selection of MSI Gene Loci

The present invention identified 22 MSI high risk loci as shown in Table 1 by referring to the database and by extensive analysis of numbers of samples from healthy human individuals and patients. The genomic location of these loci is determined from the Hg19 version of the genomic database; the “chr” and its subsequent representative number in Table 1 indicate which chromosome(s) the locus is located at.

### III. Design of the Probes

A DNA probe library was prepared for the MSI gene.

It is well-known that the specificity of capture is affected by various factors. For example, the poor design of capture probes, unsatisfactory capture conditions, insufficient blocking of repeats in genomic DNA, inappropriate ratio of genomic DNA to capture probes, and other factors can affect the specificity, sensitivity, sequencing coverage and many other aspects of the capture. In order to achieve high enrichment and low off-target rate of the target gene, a large number of experimental explorations on the probe including its type, length, sequence, hybridization conditions need to be carried out, and it is necessary to obtain the optimal parameter combination through creative exploration work. Whether or not the same effect can be achieved without the corresponding evidence is unpredictable. In the meanwhile, when a sample with mutation is being detected, the mutant proportion in the tissue sample can vary from individual to individual. Therefore, if the mutant level is low, the major problem is that the probe is unable to accurately hybridize with the mutated fragment resulting in low detection sensitivity, requiring experimental explorations on the probe sequence.

Due to the existence of complex structures in the genome such as high GC level and repetitive sequences, the sequences obtained by sequencing the final splicing assembly often cannot cover all of the sequence regions, while the uncovered part is called “Gap”. For example, a bacterial genome is sequenced with coverage of 95%, and then 5% of the sequence regions cannot be obtained by sequencing.

The probe design strategy for the MSI high-incidence loci mainly comprises: Since the microsatellite itself is a continuously repeating base sequence, placing the probe region near the microsatellite locus is likely to cause hybridization between the probes themselves, and also increases the off-target rate. Besides, the sequences with high level of A and T generally have low enrichment efficient. Therefore, one probe is added on each side of the microsatellite locus, and is only slightly overlapped with the terminals of the microsatellite locus to increase the coverage of microsatellite loci; in addition, in this design strategy, the center of the probe for MSI high-emission locus is located outside of the target site to minimize the length of consecutive repeat base sequences in the probe. In this way, the hybridization between the probes is avoided to the greatest extent, and the coverage of the target site region can also be improved. For a microsatellite locus, when the two probe regions are located at the left and right of the locus, the problem of probe hybridization caused by repeating bases at the microsatellite locus can be avoided, and in the meanwhile, a third probe covering the entire microsatellite locus is used to ensure better coverage, sequencing depth, specificity and sensitivity. In addition, some microsatellite loci have similar repetitive sequences as the genome, and need to be bypassed as much as possible. However, the probes cannot be located too far from the target site which would reduce the coverage of these microsatellite loci.

To construct a target sequence capture system based on the hybridization principle, two points are considered, namely the length and the synthesis cost of the probe. Generally, an 8-base probe has sufficient hybridization specificity and the longer the probe, the higher the specificity of hybridization. Currently, commercial kits have probe lengths between 60 nt and 200 nt. One of the important considerations is the specificity of hybridization (or mismatch tolerance for hybridization). The microsatellite locus contains a series of A or T, some of which are 20-30 bp in length and considered as repeats in the genome. If the length of the probe is too short, the probe will have a reduced specificity and an increased its off-target rate. If the length of the probe is too long, the probe is likely to form a secondary structure which is also detrimental to the enrichment efficiency. We systematically detected probes of different lengths, and finally preferred probes of 119-120 bp in length.

Illustratively, the length of the probe is finally determined to be 119-120 bases to ensure tolerance to SNPs and sensitivity to gene transversion. Through the improvement of the primer software, the designed probe is analyzed to accurately obtain the annealing temperature of the probe and the number of consecutive single bases of the GC component (such as CCCCCCC). Each probe was used to enrich and amplify the whole genome, and was screened according to the results. Each probe was separately synthesized by IDT DNA Technologies and its quality assurance was confirmed by mass spectrometry. The biotin was attached to the 5′ end of each probe for streptavidin magnetic bead enrichment.

### IV. DNA Capture Probe Hybridization

1. Hybrid the DNA Sample Library with a Biotinylated DNA Probe Library

The DNA sample library is mixed with the hybridization buffer, placed at 95° C. for 5 minutes, and then maintained at the hybridization temperature to be used. The reaction is carried out in the PCR thermocycler.

Then, the probe library is added to the mixture. The hybridization reaction is carried out in the PCR thermocycler. The mixture is incubated at 58° C., 62° C., and 65° C. respectively, and incubated at each corresponding incubation temperature for 4 hours, 8 hours, 16 hours and 24 hours respectively. In a preferred embodiment, the incubation was at 65° C. for 8 hours.

V. Obtaining the Enriched MSI-Related Gene Fragments after Hybridization

1. Prepare Streptavidin-Coated Magnetic Beads

Streptavidin magnetic beads from Dynabeads or other commercial companies are used. The beads are placed on the mixer and mixed. Each sample requires 50 μl of magnetic beads.

Magnetic bead washing: Mix 50 μl magnetic beads and 200 μl binding buffer on a mixer, separate and purify the magnetic beads from the buffer using a magnetic separator from Dynal or other commercial companies. The liquid is discarded. These steps are repeated three times, and each time 200 μl of binding buffer is added.

2. Isolation of Hybridization Product

Mix the hybridization reaction mixture from IV-1 with the streptavidin magnetic beads in V-1 and repeatedly invert the container (tube) 5 times; shake for 30 minutes at room temperature. The magnetic beads are separated and purified using a magnetic separator from Dynal or other commercial companies.

Then, 500 μl washing buffer is added to the magnetic beads. The tube is incubated at 65° C. for 10 minutes, and mixed every 5 minutes. The magnetic beads are separated and purified using a magnetic separator from Dynal or other commercial companies.

The above steps are repeated three times.

3. Release of the Enriched DNA Sample

The beads are mixed with 50 μl of elution buffer, incubated for 10 minutes at room temperature and mixed once every 5 minutes. The magnetic beads are separated using a magnetic separator from Dynal or other commercial companies and discarded. The supernatant contains a DNA sample library with enriched MSI-related gene fragments.

The sample library is purified by column provided in the commercial purification kit.

### VI. PCR Amplification and Purification

Because a certain amount of nucleic acid is lost by hybridization capture, a second amplification is needed to re-amplify the captured target fragments to meet the requirements of sequencing and quality control. This library construction method of the present invention is particularly suitable for sequencing library construction of samples with a total amount of free nucleic acid ≥10 ng or genomic DNA ≥1 μg.

The enriched DNA sample library is further amplified to prepare for sequencing.

TABLE 6
Amplification reaction composition

| Reaction material | Volume |
|---|---|
| Enriched DNA sample | ~30 μl |
| 10X High-accuracy ultra-fidelity DNA | 5 μl |
| polymerase buffer | |
| High-accuracy ultra-fidelity DNA polymerase | 1 μl |
| Forward primer | 1 μl |
| Reverse primer | 1 μl |
| Nuclease-free water | Add to make the total |
| | volume as 50 μl |

PCR conditions: placed in the PCR thermocycler, pre-denatured at 98° C. for 30 seconds, denatured at 98° C. for 30 seconds, annealed at 65° C. for 30 seconds, extended at 72° C. for 30 seconds, repeated for 4-6 times. Finally it is extended at 72° C. for 5 minutes.

The PCR amplification product is purified by column provided in the commercial purification kit.

### VII. Detection of Structure Mutations in MSI-Related Genes Using Next-Generation Sequencing Technology

Sequencing is performed using next-generation commercial sequencing instruments such as Roche 454, Illumina Hiseq, and so on. The sequencing results are analyzed using an existing sequencing software analysis package.

Illustratively, the DNA sample library template is amplified with bridge PCR using the TruSeq PE Cluster Kit v3-cBot-HS: each DNA sample fragment will form a cluster on the flow cell, generating millions of cloned clusters per lane. The Illumina HiSeq2000 next-generation sequencing system with PE-90 bp chemistry is used, which implements sequence-by-synthesis mechanism. Compared to the traditional Sanger method, the “reversible terminator reaction” technique blocks the ends of the four types of dNTP bases with a protecting group and fluorescently labels these bases with different colors.

The present invention will be further described in detail below in conjunction with specific embodiments. The examples are given only to illustrate the invention and are not intended to limit the scope of the invention.

### Example 1: Enrichment and Detection of MSI-Related Genes

First, prepare the DNA sample library to be detected

1. Extract and Fragment the ctDNA from the Plasma Sample of the Patient

1.1 DNA Extraction

A clinical blood sample of a colon cancer patient was immediately centrifuged at 2700×g for 10 min, and the upper serum was collected in a clean tube and stored at −80° C. The DNA from the peripheral blood was extracted by the QIAGEND Neasy Blood & Tissue Kit (QIAGEN, Hilden, Germany), and the DNA from the circulating tumor was extracted by the QIAamp Circulating Nucleic Acid Kit. All procedures followed the instructions in the manuals.

The quality and concentration of DNA were measured using a spectrophotometer and a gel electrophoresis system. The DNA is considered as qualified if the absorbance of DNA at 260 nm is greater than 0.05 and the ratio of absorbance A260/A280 is between 1.8 and 2.

1.2 DNA Fragmentation

Three (3) mg of high quality genomic DNA was diluted to 120 ml with low TE buffer. The DNA was fragmented according to the instructions of the tissue homogenizer, and the fragment length was 150-200 bp.

DNA was purified by column provided in the Beckman Coulter Ampure Beads kit.

1.3 DNA Sample Library Quality Detecting

Qualitative and quantitative analysis of DNA using a bioanalyzer confirm that the peak length of the DNA fragment is reasonable.

3. DNA End Repairing

With the T4 polymerase and Klenow E. coli polymerase fragments, the DNA 5′ overhang sticky ends were filled and the 3′ overhang sticky ends were flattened to produce blunt ends for subsequent blunt-end ligation. The reaction was carried out in the PCR thermocycler at 20° C. for 30 minutes.

TABLE 7
End repairing reaction composition

| Reaction material | Volume |
|---|---|
| Purified DNA sample library | 50 μl |
| Phosphorylation buffer | 10 μl |
| Deoxybase mixture dNTP (10 mM each) | 4 μl |
| T4 DNA polymerase | 5 μl |
| Klenow E. coli polymerase fragments | 1 μl |
| T4 polynucleotide kinase | 5 μl |
| Nuclease-free water | Add to make the total |
| | volume as 100 μl |

DNA was purified by column provided in the Beckman Coulter Ampure Beads kit.

4. Add Base A to the 3′ End of the DNA Sample

The reaction was carried out in the PCR thermocycler at 37° C. for 30 minutes.

TABLE 8
Reaction composition for adding A end

| Reaction material | Volume |
|---|---|
| DNA sample library | ~30 μl |
| 10X Klenow E. coli polymerase | 5 μl |
| buffer | |
| Deoxy base dATP (1 mM) | 10 μl |
| Klenow E. coli polymerase fragments | 3 μl |
| Nuclease-free water | Add to make the total |
| | volume as 50 μl |

DNA was purified by column provided in the Beckman Coulter Ampure Beads kit (Cat #: A63880).

5. Add Adapters at Both Ends of the DNA

TABLE 9
Reaction composition for adding adapters at the DNA ends

| Reaction material | Volume |
|---|---|
| DNA sample library | ~15 μl |
| 2X T4 DNA polymerase buffer | 5 μl |
| DNA adapters | 6 μl |
| T4 DNA polymerase | 3 μl |
| Nuclease-free water | Add to make the total |
| | volume as 50 μl |

DNA was purified by column provided in the Beckman Coulter Ampure Beads kit (Cat #: A63880).

6. Amplification of the DNA Sample Library from Step 5

Polymerase chain reaction (PCR) was performed in the PCR thermocycler.

TABLE 10
PCR reaction composition

| Reaction material | Volume |
|---|---|
| DNA sample library with adapters added | ~30 μl |
| 10X High-accuracy ultra-fidelity DNA | 5 μl |
| polymerase buffer | |
| High-accuracy ultra-fidelity DNA polymerase | 1 μl |
| Adapter forward primer | 1 μl |
| Adapter reverse primer | 1 μl |
| Nuclease-free water | Add to make the total |

PCR conditions: placed in the PCR thermocycler, pre-denatured at 98° C. for 30 seconds, denatured at 98° C. for 30 seconds, annealed at 65° C. for 30 seconds, extended at 72° C. for 30 seconds, repeated 4-6 times (DNA sample library). Finally, it was extended at 72° C. for 5 minutes.

PCR amplification product was purified by column provided in the Beckman Coulter Ampure Beads kit (Cat #: A63880).

9. Quality Analysis of the DNA Sample Library after Amplification

DNA biochemical quantitative analysis was performed using a bioanalyzer, which confirmed that the peak length of the fragment after purification is reasonable, about 200 bp. Therefore, a ctDNA sample library was obtained.

For the obtained DNA sample library, if the DNA concentration is less than 150 ng/μl, the sample must be dried at a low temperature (lower than 45° C.) by a vacuum concentrator, and then dissolved in the nuclease-free water to the desired concentration. The enrichment and detection of the obtained whole-genome DNA sample library will be carried out for this embodiment.

### II. Preparation of DNA Probe Library According to the MSI Loci

The probes were designed, synthesized and labeled with biotin at the 5′ end following the design method and strategy mentioned above.

III. Hybrid the DNA Sample Library with a Biotinylated DNA Probe Library

The DNA sample library was mixed with the hybridization buffer (SeqCap Hybridization and wash kit from Nimblegen) (the final DNA sample concentration in the mixture was ≤50 ng/μl), placed at 95° C. for 5 minutes, and then maintained at the hybridization temperature to be used. The reaction was carried out in the PCR thermocycler.

Then, 3 pmol of probe library was added to the mixture and incubated at 65° C. for 5 min. The hybridization reaction was carried out in the PCR thermocycler. The mixture was incubated at 65° C. for 8 hours.

IV. Obtaining the Enriched MSI-Related Gene Fragments after Hybridization

1. Prepare Streptavidin-Coated Magnetic Beads

Streptavidin magnetic beads from Dynabeads (Life technologies, Cat #: 11206D) or other commercial companies were used. The beads were placed on the mixer and mixed.

Magnetic bead washing: Mix 50 μl magnetic beads and 200 μl binding buffer (SeqCap Hybridization and wash kit by Nimblegen) on a mixer, separate and purify the magnetic beads from the buffer using a magnetic separator from Dynal or other commercial companies. The liquid was discarded. These steps were repeated three times, and each time 200 μl of binding buffer was added.

2. Isolation of Hybridization Product

Mix the hybridization reaction mixture from IV with the streptavidin magnetic beads in step 1 of V and repeatedly invert the container (tube) 5 times; shake for 30 minutes at room temperature. The magnetic beads were separated and purified using a magnetic separator from Dynal or other commercial companies.

Then, 500 μl washing buffer was added to the magnetic beads. The tube was incubated at 65° C. for 10 minutes, and mixed every 5 minutes. The magnetic beads were separated and purified using a magnetic separator from Dynal or other commercial companies. These steps were repeated three times.

3. Release of the Enriched DNA Sample

The beads were mixed with 50 μl of elution buffer (10 mM NaOH), incubated for 10 minutes at room temperature and mixed once every 5 minutes. The magnetic beads were separated using a magnetic separator from Dynal or other commercial companies and then discarded. The supernatant contained a DNA sample library with enriched MSI-related gene fragments.

The sample library was purified by column provided in the Beckman Coulter Ampure Beads kit (Cat #: A63880).

### V. PCR Amplification and Purification

The enriched DNA sample library was further amplified to prepare for sequencing.

TABLE 11
Amplification reaction composition

| Reaction material | Volume |
|---|---|
| Enriched DNA sample | ~30 μl |
| 10X High-accuracy ultra-fidelity DNA | 5 μl |
| polymerase buffer | |
| High-accuracy ultra-fidelity DNA polymerase | 1 μl |
| Forward primer | 1 μl |
| Reverse primer | 1 μl |
| Nuclease-free water | Add to make the total |
| | volume as 50 μl |

PCR conditions: placed in the PCR thermocycler, pre-denatured at 98° C. for 30 seconds, denatured at 98° C. for 30 seconds, annealed at 65° C. for 30 seconds, extended at 72° C. for 30 seconds, repeated for 4-6 times. It was extended at 72° C. for 5 minutes at the final step.

The PCR amplification product was purified by column provided in the Beckman Coulter Ampure Beads kit (Cat #: A63880).

### VI. Detection of Structure Mutations in MSI-Related Genes Using Next-Generation Sequencing Technology

The DNA sample library template was amplified with bridge PCR using the TruSeq PE Cluster Kit v3-cBot-HS: each DNA sample fragment will form a cluster on the flowcell, generating millions of cloned clusters per lane. The Illumina HiSeq4000 next-generation sequencing system with PE-150 bp chemistry is used, which implements sequence-by-synthesis mechanism. Compared to the traditional Sanger method, the “reversible terminator reaction” technique blocks the ends of the four types of dNTP bases with a protecting group and fluorescently labels these bases with different colors.

After QC screening, the sequencing results were mapped with Bowtie for the obtained fragments.

Fragments for mutation analysis were successfully mapped using the Bioconductor software.

According to the above method, the detection results using different probes are as follows:

The probe length has a great influence on the specificity and target rate of the detection. Therefore, probes of different lengths are designed under the condition of 2 times of coverage. We designed probes of three different lengths, 100 nt, 120 nt and 140 nt, and determined the optimal length based on the target rate and cost.

The results of three repeated detections were as follows:

TABLE 12
Comparison of detection effects for probes with different length

| | | | | Mean of | | |
|---|---|---|---|---|---|---|
| Probe | | | | target | | |
| length | Target rate | | | rate | Statistic analysis | |
| 100 nt | 34.54% | 38.23% | 33.48% | 35.42% | p < 0.05 | |
| 140 nt | 52.34% | 48.76% | 53.32% | 51.47% | | P < 0.05 |
| 120 nt | 67.75% | 65.69% | 68.21% | 67.22% | | |

According to the sequencing results, the target rate was the highest with the 120 nt probe and the lowest with the 100 nt probe. In particular, the 140 nt probe showed a significant better target rate than the 100 nt probe; the 120 nt probe showed a significant better target rate than to the 140 nt probe. Therefore, the 120 nt probe is optimal for high target rate.

The final length of the probe was determined as 120 bases, which ensures the target rate for capturing the target sequence. The designed probe was analyzed with upgraded Primer software to accurately determine the annealing temperature of the probe and the number of consecutive single bases of the GC component (e.g. CCCCCCC).

In addition, three sets of experiments were designed to determine the optimal probe concentration. The experimental results using a 120 nt length probe were as follows:

TABLE 13
Comparison of detection effects for probes at different concentrations

| | | | | Mean | | |
|---|---|---|---|---|---|---|
| Probe | | | | target | | |
| concentration | 1 | 2 | 3 | rate | Statistic analysis | |
| 1 pmol | 34.32% | 42.53% | 39.57% | 38.81% | p < | |
| 3 pmol | 68.15% | 66.69% | 67.21% | 67.35% | 0.05 | p > 0.05 |
| 6 pmol | 61.34% | 58.77% | 60.43% | 60.18% | | |

According to the sequencing results, the use of 1 pmol probe resulted in a significant lower target rate than the 3 pmol probe. The use of 6 pmol probe leaded to a slight lower target rate than the 3 pmol probe, but showed a significant higher target rate than the 1 pmol probe.

We also analyzed the sequencing depth of the target DNA sequence for each probe, and the results were as follows:

TABLE 14
Comparison of coverage by probes at different concentrations

| | Coverage | | | |
|---|---|---|---|---|
| Probe | ratio for | >0.2x average | >0.5x average | >1x average |
| concen- | target | coverage | coverage | coverage |
| tration | region | percentage | percentage | percentage |
| 1 pmol | 34.5 | 93.7% | 46.9% | 33.2% |
| 3 pmol | 62.5 | 99.7% | 91.5% | 51.3% |
| 6 pmol | 60.3 | 99.1% | 90.7% | 50.2% |

Note:
>0.2x average coverage percentage means the proportion of the regions which has a depth of coverage higher than 20% of the average depth of coverage for the target DNA sequence, against the total region. Similarly, >0.5x and >1x average coverage percentage mean the proportion of regions with a depth of coverage greater than 50% and 100% respectively of the average depth of coverage of all target region.

According to the sequencing results, as the concentration of the probe increases, the average coverage ratio of the target region gradually increases, and the uniformity improves. Considering the target rate and cost, we chose the 3 pmol probe as the coverage ratio of the exon region.

While a probe is being designed, many factors should be taken into consideration including the large number of microsatellite loci that need to be captured and the presence of repeating bases in the microsatellite loci which cause hybridization between probes and affect the probe specificity, coverage of target sequences, and sensitivity. Therefore, a lot of exploratory trials are required for the probe design.

Finally, due to the sequence specificity of each microsatellite locus, the efficiency of enrichment for each microsatellite locus will be different, as reflected in the homogeneity of targeted enrichment. To enhance uniformity of coverage, probes used for enriching each microsatellite locus were first mixed, enriched and sequenced in equimolar proportions. Based on the sequencing results, we adjusted the ratio of the probes by increasing the proportion of probes for loci with lower coverage and reducing the proportion of probes for loci with excessive coverage. After several rounds of optimization of the probe ratio, we made the coverage ratio of all microsatellite sites uniform and consistent.

The detection sensitivity for microsatellite instability also needs to be investigated experimentally. We constructed a QC plasmid for the PKHD-18 locus.

Positive control plasmid: A plasmid for constructing a PKHD-18 site deletion mutation.

Internal control plasmid: a wild-type plasmid of the same sequence corresponding to the above positive plasmid.

The positive control plasmid and the internal control plasmid were mixed according to the copy number ratio to obtain a plasmid sample solution with different deletion mutation frequencies, and then the DNA concentration was adjusted with Tris-HCl buffer (10 mM, pH 8.5) to reach a final DNA concentration as 5 μg/μl in the solution.

In the probe design for the PKHD-18 site, the following probes were used:

TABLE 15
Comparison of different probes

| Probe group | Probe sequence |
|---|---|
| 1 | SEQ ID NO. 67 |
| | SEQ ID NO. 68 |
| | SEQ ID NO. 69 |
| 2 | SEQ ID NO. 70 |
| | SEQ ID NO. 71 |
| | SEQ ID NO. 72 |
| 3 | SEQ ID NO. 73 |
| | SEQ ID NO. 74 |
| | SEQ ID NO. 75 |
| 4 | SEQ ID NO. 49 |
| | SEQ ID NO. 50 |
| | SEQ ID NO. 51 |

The detection results from the above four groups of probes are shown as below:

| | 5% plasmid | | | 2% plasmid | | | 1% plasmid | | |
|---|---|---|---|---|---|---|---|---|---|
| Group 1 | 6.98% | 8.44% | 4.42% | — | — | 1.53% | — | — | — |
| Group 2 | 5.21% | 4.21% | 4.22% | — | 1.94% | 1.36% | — | — | — |
| Group 3 | 5.32% | 3.56% | 4.867% | 1.98% | 1.43% | 1.36% | 0.76% | — | — |
| Group 4 | 4.19% | 5.54% | 4.82% | 1.87% | 2.13% | 2.26% | 0.95% | 1.15% | 0.84% |

As can be seen, when a preferred probe was used, a 1% abundance deletion mutation could be detected, indicating high sensitivity. In addition, the above mutations were verified by the ARMS-PCR method.

Finally, the preferred probe sequences were determined as SEQ ID NOS. 1-66.

TABLE 16
Preferred probe sequence for each microsatellite locus

| Locus | Microsatellite | |
|---|---|---|
| number | locus name | Probe sequence |
| MS-1 | BAT25 | SEQ ID NO. 1-3 |
| MS-2 | BAT26 | SEQ ID NO. 4-6 |
| MS-3 | NR24 | SEQ ID NO. 7-9 |
| MS-4 | NR21 | SEQ ID NO. 10-12 |
| MS-5 | Mono27 | SEQ ID NO. 13-15 |
| MS-6 | NR22 | SEQ ID NO. 16-18 |
| MS-7 | NR27 | SEQ ID NO. 19-21 |
| MS-8 | BAT40 | SEQ ID NO. 22-24 |
| MS-9 | CUL-22 | SEQ ID NO. 25-27 |
| MS-10 | MET-15 | SEQ ID NO. 28-30 |
| MS-11 | ATM-15 | SEQ ID NO. 31-33 |
| MS-12 | RB1-13 | SEQ ID NO. 34-36 |
| MS-13 | NF1-26 | SEQ ID NO. 37-39 |
| MS-14 | DDR-11 | SEQ ID NO. 40-42 |
| MS-15 | FANC-21 | SEQ ID NO. 43-45 |
| MS-16 | MITF-14 | SEQ ID NO. 46-48 |
| MS-17 | PKHD-18 | SEQ ID NO. 49-51 |
| MS-18 | PTK-16 | SEQ ID NO. 52-54 |
| MS-19 | RET-14 | SEQ ID NO. 55-57 |
| MS-20 | CBL-17 | SEQ ID NO. 58-60 |
| MS-21 | PTPN-17 | SEQ ID NO. 61-63 |
| MS-22 | SMAD-18 | SEQ ID NO. 64-66 |

To further determine the sequence capture effect and the high-throughput sequencing condition of the probes, the IHC technology, the PCR technology and the preferred probe technology of the present invention were performed simultaneously using the MSI-H cell line (HCT116) and 26 tumor patient samples. The results using different platforms were compared: cell line verification results are shown in FIG. 3, PCR detection and the probe detection of the present invention are shown as MSI-H; verification results for 26 tumor patients are shown in Table 17.

TABLE 17
Detection results using NGS, PCR and IHC methods

| | PCR | | | | | | |
|---|---|---|---|---|---|---|---|
| | detection | IHC detection | | | | | NGS detection |
| Sample number | result | MLH1 | MSH2 | MSH6 | PMS2 | MMR result | result |
| 16 . . . 11 | MSI-H | + | + | − | + | dMMR | MSI-H |
| 16 . . . 13 | MSI-H | − | + | + | − | dMMR | MSI-H |
| 16 . . . 15 | MSI-H | − | + | + | − | dMMR | MSI-H |
| 16 . . . 17 | MSI-H | + | + | − | − | dMMR | MSI-H |
| 16 . . . 19 | MSI-H | + | + | + | − | dMMR | MSI-H |
| 16 . . . 21 | MSI-H | − | + | + | − | dMMR | MSI-H |
| 16 . . . 23 | MSS | + | − | − | + | dMMR | MSS |
| 16 . . . 25 | MSI-H | + | − | − | + | dMMR | MSI-H |
| 16 . . . 27 | MSI-H | + | − | + | + | dMMR | MSI-H |
| 16 . . . 29 | MSI-L | + | + | + | − | dMMR | MSI-L |
| 16 . . . 31 | MSI-H | + | + | + | − | dMMR | MSI-H |
| 16 . . . 33 | MSI-H | + | + | − | − | dMMR | MSI-H |
| 16 . . . 35 | MSI-H | + | + | + | − | dMMR | MSI-H |
| 16 . . . 37 | MSS | + | + | − | + | dMMR | MSS |
| 16 . . . 39 | MSI-H | − | + | + | − | dMMR | MSI-H |
| 16 . . . 41 | MSI-H | − | + | + | − | dMMR | MSI-H |
| 16 . . . 43 | MSI-L | + | + | − | + | dMMR | MSI-L |
| 16 . . . 45 | MSI-L | + | + | − | + | dMMR | MSI-L |
| 16 . . . 47 | MSI-H | + | − | − | + | dMMR | MSI-H |
| 16 . . . 49 | MSI-H | + | − | − | + | dMMR | MSI-H |
| 16 . . . 51 | MSS | + | + | + | + | pMMR | MSS |
| 16 . . . 53 | MSS | + | + | + | + | pMMR | MSS |
| 16 . . . 55 | MSS | + | + | + | + | pMMR | MSS |
| 16 . . . 57 | MSS | + | + | + | + | pMMR | MSS |
| 16 . . . 59 | MSS | + | + | + | + | pMMR | MSS |
| 16 . . . 61 | MSI-H | − | + | + | − | dMMR | MSI-H |

TABLE 18
Detection results of 22 MSI-related loci for each sample

| Sample number | MSI-1 | MSI-2 | MSI-3 | MSI-4 | MSI-5 | MSI-6 | MSI-7 | MSI-8 | MSI-9 | MSI-10 | MSI-11 |
|---|---|---|---|---|---|---|---|---|---|---|---|
| 16 . . . 11 | − | − | + | + | + | − | − | + | + | − | + |
| 16 . . . 13 | − | − | + | + | + | + | − | + | − | − | + |
| 16 . . . 15 | − | + | + | + | + | − | − | + | − | − | − |
| 16 . . . 17 | − | + | + | − | + | − | − | + | + | − | + |
| 16 . . . 19 | − | − | + | − | + | − | − | + | + | − | + |
| 16 . . . 21 | − | + | + | + | + | + | − | − | + | − | + |
| 16 . . . 23 | − | − | − | − | − | − | − | − | + | − | − |
| 16 . . . 25 | − | + | − | + | − | + | − | + | + | − | + |
| 16 . . . 27 | − | − | − | + | − | − | − | + | + | − | + |
| 16 . . . 29 | − | − | + | − | + | − | − | + | − | − | + |
| 16 . . . 31 | − | + | + | + | + | + | − | + | + | − | − |
| 16 . . . 33 | − | − | + | − | − | + | − | + | + | − | + |
| 16 . . . 35 | − | − | + | + | + | + | − | + | + | − | + |
| Sample number | MSI-12 | MSI-13 | MSI-14 | MSI-15 | MSI-16 | MSI-17 | MSI-18 | MSI-19 | MSI-20 | MSI-21 | MSI-22 |
| 16 . . . 11 | + | − | + | + | + | − | − | − | + | + | + |
| 16 . . . 13 | + | − | + | + | − | − | − | − | − | − | + |
| 16 . . . 15 | + | + | + | + | + | + | − | + | + | + | − |
| 16 . . . 17 | + | + | + | + | + | − | + | − | + | + | + |
| 16 . . . 19 | − | − | − | + | − | − | − | − | + | + | + |
| 16 . . . 21 | + | + | + | + | + | + | − | − | + | − | + |
| 16 . . . 23 | − | − | − | − | + | − | − | − | − | − | − |
| 16 . . . 25 | + | − | + | + | + | − | + | − | + | + | + |
| 16 . . . 27 | + | + | + | + | − | + | + | − | + | − | + |
| 16 . . . 29 | − | − | + | + | − | − | − | − | + | + | − |
| 16 . . . 31 | + | + | + | + | + | + | − | + | + | + | − |
| 16 . . . 33 | − | − | + | + | + | + | + | − | + | + | + |
| 16 . . . 35 | + | − | + | + | + | + | + | + | − | + | + |
| Sample number | MSI-1 | MSI-2 | MSI-3 | MSI-4 | MSI-5 | MSI-6 | MSI-7 | MSI-8 | MSI-9 | MSI-10 | MSI-11 |
| 16 . . . 37 | − | − | − | − | − | − | − | + | − | − | − |
| 16 . . . 39 | − | + | + | − | + | + | − | − | − | − | + |
| 16 . . . 41 | − | + | + | + | + | − | − | + | + | − | + |
| 16 . . . 43 | − | − | − | + | + | − | − | + | − | − | + |
| 16 . . . 45 | − | − | − | − | − | − | − | + | + | + | + |
| 16 . . . 47 | − | − | + | − | + | − | − | + | + | − | + |
| 16 . . . 49 | − | − | + | + | + | − | − | + | + | + | + |
| 16 . . . 51 | − | − | − | − | − | − | − | − | − | − | − |
| 16 . . . 53 | − | − | − | − | − | − | − | − | − | − | − |
| 16 . . . 55 | − | − | − | − | − | − | − | − | − | − | − |
| 16 . . . 57 | − | − | − | − | − | − | − | − | − | − | − |
| 16 . . . 59 | − | − | − | − | − | − | − | − | − | − | − |
| 16 . . . 61 | − | − | + | + | + | + | − | + | + | + | + |
| Sample number | MSI-12 | MSI-13 | MSI-14 | MSI-15 | MSI-16 | MSI-17 | MSI-18 | MSI-19 | MSI-20 | MSI-21 | MSI-22 |
| 16 . . . 37 | − | − | + | − | − | − | − | − | − | − | − |
| 16 . . . 39 | + | + | + | + | − | + | + | − | + | + | + |
| 16 . . . 41 | + | − | + | + | + | + | − | − | − | + | − |
| 16 . . . 43 | − | − | + | − | − | + | − | − | + | − | − |
| 16 . . . 45 | − | − | − | − | − | − | − | − | − | − | − |
| 16 . . . 47 | + | − | + | + | − | + | − | + | − | + | − |
| 16 . . . 49 | + | + | + | + | + | + | + | + | + | − | + |
| 16 . . . 51 | − | − | − | − | − | − | − | − | − | − | − |
| 16 . . . 53 | − | − | − | − | − | − | − | − | − | − | − |
| 16 . . . 55 | − | − | − | − | − | − | − | − | − | − | − |
| 16 . . . 57 | − | − | + | − | − | − | − | − | − | − | − |
| 16 . . . 59 | − | − | − | − | − | − | − | − | − | − | − |
| 16 . . . 61 | + | + | + | + | + | + | + | − | + | − | + |

The detection results using the probes of the present invention are highly consistent with those using PCR and IHC. The sensitivity and specificity of the preferred probe technology provided by the present invention are 100% compared to the PCR detection; the sensitivity is 90.5% and the specificity is 100% compared to the IHC technology.

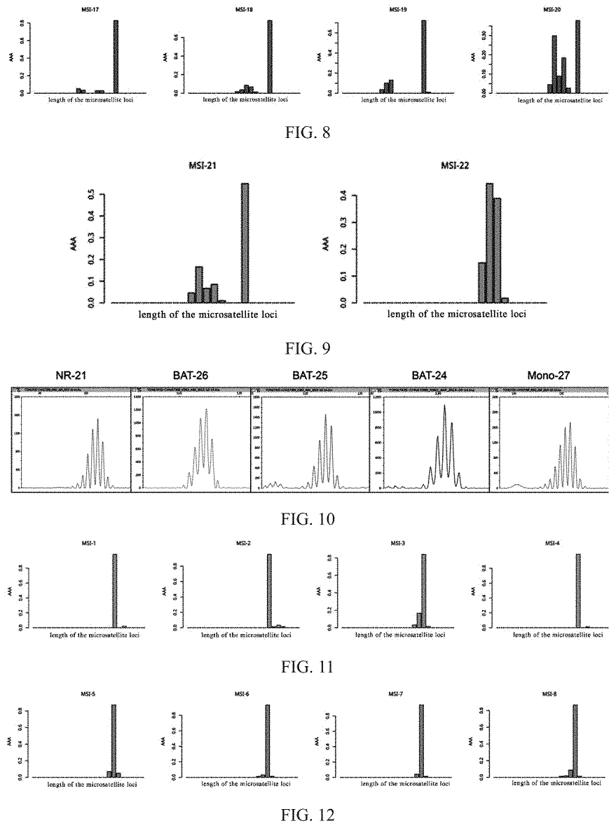

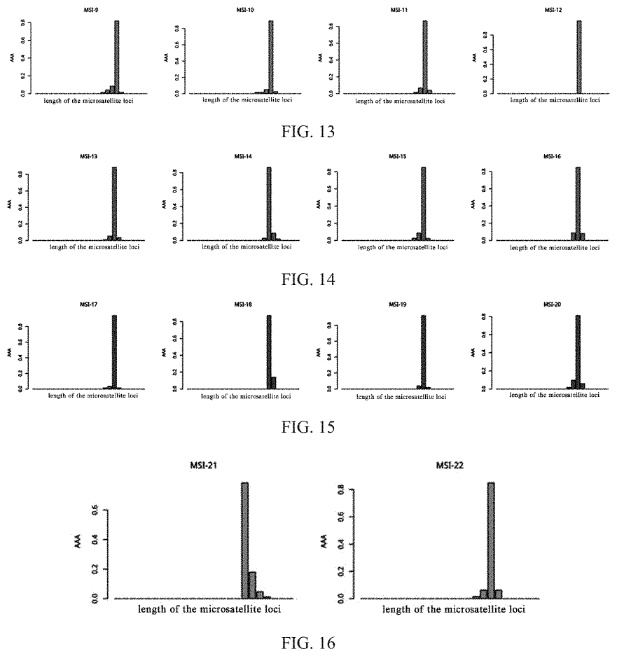

The PCR detection data and the present invention detection data for one case of MSI-H and MSS are shown in FIG. 3-9 (MSI-H) and FIG. 10-16 (MSS) respectively. All data demonstrated that the method of the present invention is accurate and reliable for above MSI detection.

What is claimed is:

1. A DNA probe library for a hybridization with microsatellite instability (MSI)-related microsatellite loci, wherein
the DNA probe library is configured to be hybridized to 22 different microsatellite loci with mononucleotide repeats in a human genome, wherein the 22 different microsatellite loci comprise BAT25, BAT26, NR24, NR21, Mono27, NR22, NR27, BAT40, CUL-22, MET-15, ATM-15, RB1-13, NF1-26, DDR-11, FANC-21, MITF-14, PKHD-18, PTK-16, RET-14, CBL-17, PTPN-17, and SMAD-18; wherein positions of the 22 different microsatellite loci in the human genome are:
chr4:55,598,212-55,598,236,
chr2:47,641,560-47,641,586,
chr2:95,849,362-95,849, 384,
chr14:23,652,347-23,652,367,
chr2:39,536,690-39,536,716,
chr11:125,490,766-125,490,786,
chr11:102,193,509-102,193,534,
chr1:120,053,341-120,053,377,
chr2:225,422,601-225,422,622,
chr7:116,409,676-116,409,690,
chr11:108,114,662-108,114,676,
chr13:48,954,160-48,954,172,
chr17:29,559,062-29,559,087,
chr1:162,736,822-162,736,832,
chr3:10,076,009-10,076,029,
chr3:69,988,438-69,988,451,
chr6:51,503,598-51,503,615,
chr8:141,754,889-141,754,904,
chr10:43,595,837-43,595,850,
chr11:119,144,792-119,144,808,
chr12:112,893,676-112,893,692, and
chr18:45,395,846-45,395,863, respectively, in human reference genome hg19.

2. The DNA probe library for the hybridization with the microsatellite instability (MSI)-related microsatellite loci according to claim 1, comprising 22 sets of a first probe, a second probe and a third probe, wherein the 22 sets of the first probe, the second probe and the third probe are respectively designed for the corresponding 22 different microsatellite loci, wherein
the first probe has one end located at an upstream of a sequence of the corresponding microsatellite locus and an other end located inside the corresponding microsatellite locus,
the second probe has one end located inside the corresponding microsatellite locus and an other end located a downstream of the sequence of the corresponding microsatellite locus,
the third probe has two ends having regions specifically binding to the corresponding microsatellite locus and the two ends of the third probe are located an upstream and a downstream of the corresponding microsatellite locus respectively; and
the DNA probe library comprises at least one of probes having nucleotide sequences as shown in SEQ ID NOS: 1-66.

3. A method for enriching fragments of MSI-related microsatellite loci, comprising
1) obtaining a DNA sample library from a subject;
2) obtaining the DNA probe library according to claim 1;
3) hybridizing the DNA probe library with the DNA sample library to obtain a hybridization product; and
4) isolating the hybridization product of the step 3) to obtain hybridization-enriched fragments, followed by performing a release of the hybridization-enriched fragments of the MSI-related microsatellite loci to obtain the fragments of the MSI-related microsatellite loci.

4. The method for enriching the fragments of the MSI-related microsatellite loci according to claim 3, wherein the DNA sample library in the step 1) consists of double-stranded DNA fragments and the step 1) further comprises extracting a whole genomic DNA from the subject and then fragmenting the whole genomic DNA.

5. The method for enriching the fragments of the MSI-related microsatellite loci according to claim 3, wherein the subject is a human, and a whole genomic DNA is extracted from cell, tissue or body fluid samples of the subject; DNA fragments are 150-600 bp in length.

6. The method for enriching the fragments of the MSI-related microsatellite loci according to claim 3, wherein the step 3) comprises:
a) labeling DNA probes in the DNA probe library with selectable markers; and
b) hybridizing the DNA probe library with the DNA sample library;
the selectable markers in the step a) are biotin; the step b) comprises incubating the DNA probe library with the DNA sample library for 24 hours at 65° C. in a PCR thermocycler; and
in the step 4) of the method, the hybridization product is isolated by the selectable markers on the DNA probes, the selectable markers in the step a) are biotin, and in the step 4) the hybridization product is isolated by an affinity of streptavidin-biotin.

7. A method for detecting a change in number of repeat units in MSI-related microsatellite loci, comprising:
A) enriching the fragments of the MSI-related microsatellite loci according to the method of claim 3; and
B) detecting the change in the number of the repeat units in the MSI-related microsatellite loci.

8. The method for detecting the change in the number of the repeat units in the MSI-related microsatellite loci according to claim 7, wherein, in the step B), through next-generation sequencing technology (NGS), the enriched fragments of the MSI-related microsatellite loci are sequenced to detect the change in the number of the repeat units in the MSI-related microsatellite loci.

9. A kit for enriching fragments of MSI-related microsatellite loci, comprising the DNA probe library of claim 1.

10. Use of the kit of claim 9 for detecting instability of microsatellite instability (MSI) related microsatellite loci for non-therapeutic and non-diagnostic purposes.


SEQUENCE LISTING (75 sequences)

SEQ ID NO: 1 - DNA, 120 bp, Artificial sequence
  The sequence is synthesized
agacttgctgagcttttcttaccaggtggcaaagggcatggctttcctcgcctccaagaatgtaagtgggagtgattctctaaagagttttgtgttttgtttttttgatttttttttttt

SEQ ID NO: 2 - DNA, 120 bp, Artificial sequence
  The sequence is synthesized
tttttttttttttgagaacagagcattttagagccatagttaaaatgcagaatgtcattttgaagtgtggtaaccaaaagcagaggaaatttagtttcttcatgttccaactgctgtctc

SEQ ID NO: 3 - DNA, 120 bp, Artificial sequence
  The sequence is synthesized
aagaatgtaagtgggagtgattctctaaagagttttgtgttttgtttttttgatttttttttttttttttttttttttgagaacagagcattttagagccatagttaaaatgcagaatgt

SEQ ID NO: 4 - DNA, 120 bp, Artificial sequence
  The sequence is synthesized
ttatcagatgattccaactttggacagtttgaactgactacttttgacttcagccagtatatgaaattggatattgcagcagtcagagcccttaacctttttcaggtaaaaaaaaaaaaa

SEQ ID NO: 5 - DNA, 120 bp, Artificial sequence
  The sequence is synthesized
aaaaaaaaaaaaaagggttaaaaatgttgaatggttaaaaaatgttttcattgacatatactgaagaagcttataaaggagctaaaatattttgaaatattattatacttggattagata

SEQ ID NO: 6 - DNA, 120 bp, Artificial sequence
  The sequence is synthesized
agtatatgaaattggatattgcagcagtcagagcccttaacctttttcaggtaaaaaaaaaaaaaaaaaaaaaaaaaaagggttaaaaatgttgaatggttaaaaaatgttttcattgac

SEQ ID NO: 7 - DNA, 120 bp, Artificial sequence
  The sequence is synthesized
atttagtgaaaaattatctgaatatttaaggtctgccttaacgtgatccccattgctgaattttacctcctgactccaaaaactcttctcttccctgggcccagtcctattttttttttt

SEQ ID NO: 8 - DNA, 120 bp, Artificial sequence
  The sequence is synthesized
ttttttttttttgtgagacagagtctcactctgtcacccaggttggaatgcaatggcacaatctccgctcactgcaagctccgcctcccgggttcacgccattctcctgcctcagcctcc

SEQ ID NO: 9 - DNA, 120 bp, Artificial sequence
  The sequence is synthesized
ctgaattttacctcctgactccaaaaactcttctcttccctgggcccagtcctatttttttttttttttttttttttgtgagacagagtctcactctgtcacccaggttggaatgcaatg

SEQ ID NO: 10 - DNA, 120 bp, Artificial sequence
  The sequence is synthesized
actgctactctctaaaaaaggcaagcagataaaagagaacacgaaaaatattcctactccgcattcacactttctggtcactcgcgtttacaaacaagaaaagtgttgctaaaaaaaaaa

SEQ ID NO: 11 - DNA, 120 bp, Artificial sequence
  The sequence is synthesized
aaaaaaaaaaaggccaggggagacatacatttaaatataaaaatagaactgtgccagcgactccggctggaattctgctgaaagggatgtgtcttcagaaaccaggatggttctgctctg

SEQ ID NO: 12 - DNA, 120 bp, Artificial sequence
  The sequence is synthesized
actccgcattcacactttctggtcactcgcgtttacaaacaagaaaagtgttgctaaaaaaaaaaaaaaaaaaaaaggccaggggagacatacatttaaatataaaaatagaactgtgcc

SEQ ID NO: 13 - DNA, 120 bp, Artificial sequence
  The sequence is synthesized
ctaaggctatactacttacaagttcatgatgtggttctgtctcctttcttaagggtggatcaaatttcacttggccaactaaaaaagaaaaaaaagtaaaaccaggattttttttttttt

SEQ ID NO: 14 - DNA, 120 bp, Artificial sequence
  The sequence is synthesized
ttttttttttttttgaggcagagtcttgctctgtctcccacgctggagtgcagtggcgcaatctcagctcactgcaatctccaccccctgggttcccaccatttccctgcctcagcctcc

SEQ ID NO: 15 - DNA, 120 bp, Artificial sequence
  The sequence is synthesized
tggatcaaatttcacttggccaactaaaaaagaaaaaaaagtaaaaccaggatttttttttttttttttttttttttttgaggcagagtcttgctctgtctcccacgctggagtgcagtg

SEQ ID NO: 16 - DNA, 120 bp, Artificial sequence
  The sequence is synthesized
ttatgttgcaggtaaaggacctggataatcgaggcttgtcaaggacataaatgtcacgtccagctctgatatgcttcgcactgagcacatcacatttaggacgttgaagatttttttttt

SEQ ID NO: 17 - DNA, 120 bp, Artificial sequence
  The sequence is synthesized
tttttttttttaatatgcagtttgtaagaacaaaactggatggcatcagaattgtctggaagttttgtcttgggcagtatgggctgggccaaatgaaatgatttttataattctaaacag

SEQ ID NO: 18 - DNA, 120 bp, Artificial sequence
  The sequence is synthesized
acgtccagctctgatatgcttcgcactgagcacatcacatttaggacgttgaagatttttttttttttttttttttaatatgcagtttgtaagaacaaaactggatggcatcagaattgt

SEQ ID NO: 19 - DNA, 120 bp, Artificial sequence
  The sequence is synthesized
atgagatgtgatgtttctcctgccacctggaaacaaagcattgaagtctgcagttgaaaagcccaacgtctgtgagatccaggaaaccatgcttgcaaaccactggtaaaaaaaaaaaaa

SEQ ID NO: 20 - DNA, 120 bp, Artificial sequence
  The sequence is synthesized
aaaaaaaaaaaaagccacagtgacttgcttattggtcattgctagtattatcgactcagaacctctttactaatggctagtaaatcataattgagaaattctgaattttgacaaggtctc

SEQ ID NO: 21 - DNA, 120 bp, Artificial sequence
  The sequence is synthesized
gaaaagcccaacgtctgtgagatccaggaaaccatgcttgcaaaccactggtaaaaaaaaaaaaaaaaaaaaaaaaaagccacagtgacttgcttattggtcattgctagtattatcgac

SEQ ID NO: 22 - DNA, 120 bp, Artificial sequence
  The sequence is synthesized
ctcaaatcccctataccagtccattttatatcctcaagccaagattaacttcctacaccacaaccctgcttttgttcctttttttgttgttgcttgtgtggttttctttttttttttttt

SEQ ID NO: 23 - DNA, 120 bp, Artificial sequence
  The sequence is synthesized
tttttttttttttttttttttttgagacaaggtggtcttgctctaccacccaggctagagtgcagaggtgtgatcttggctcactgccaggaaccagagaccccagcctccatttatcct

SEQ ID NO: 24 - DNA, 120 bp, Artificial sequence
  The sequence is synthesized
caaccctgcttttgttcctttttttgttgttgcttgtgtggttttctttttttttttttttttttttttttttttttttttttgagacaaggtggtcttgctctaccacccaggctagag

SEQ ID NO: 25 - DNA, 120 bp, Artificial sequence
  The sequence is synthesized
accactgttattcttacgctggatttcttgaattgcatttttcagaaggtcccaaatgctgtttacatatttttcatccatggtcatctgtaatatccaagagagagaagagacaaaaaa

SEQ ID NO: 26 - DNA, 120 bp, Artificial sequence
  The sequence is synthesized
aaaaaccaatggttgaaagtctttctatatttgtccatatatagtaaatattgtattatatagatatgattataaatctatgacacataagaacattaacaacctattttaacaggggga

SEQ ID NO: 27 - DNA, 120 bp, Artificial sequence
  The sequence is synthesized
ttacatatttttcatccatggtcatctgtaatatccaagagagagaagagacaaaaaaaaaaaaaaaaaaaaaaccaatggttgaaagtctttctatatttgtccatatatagtaaatat

SEQ ID NO: 28 - DNA, 120 bp, Artificial sequence
  The sequence is synthesized
ttagtatcatagaatcgtgtgccttggcaaacaacatggcctgtgtttgcagtatatttatattcctttgccattgttagcattcctgcagaactgtgaagtgttaacaacctttttttt

SEQ ID NO: 29 - DNA, 120 bp, Artificial sequence
  The sequence is synthesized
ttttttttcctttcagggaaatgatattgaccctgaagcagttaaaggtgaagtgttaaaagttggaaataagagctgtgagaatatacacttacattctgaagccgttttatgcacggt

SEQ ID NO: 30 - DNA, 120 bp, Artificial sequence
  The sequence is synthesized
tattcctttgccattgttagcattcctgcagaactgtgaagtgttaacaacctttttttttttttttcctttcagggaaatgatattgaccctgaagcagttaaaggtgaagtgttaaaa

SEQ ID NO: 31 - DNA, 120 bp, Artificial sequence
  The sequence is synthesized
ttaaaaataaattagtgcagttttaaaatcctttttctgtatgggattatggaatatttaagttaaattgtaacatttaatacattttgatttttaaaaaatcatgactaataatttttt

SEQ ID NO: 32 - DNA, 120 bp, Artificial sequence
  The sequence is synthesized
ttttttaagaattgttctctgtgtacttcaggctctatctgaaaccttcacaagatgttcatagagttttagtggctagaataattcatgctgttaccaaaggatgctgttctcagactg

SEQ ID NO: 33 - DNA, 120 bp, Artificial sequence
  The sequence is synthesized
gttaaattgtaacatttaatacattttgatttttaaaaaatcatgactaataatttttttttttttttaagaattgttctctgtgtacttcaggctctatctgaaaccttcacaagatgt

SEQ ID NO: 34 - DNA, 120 bp, Artificial sequence
  The sequence is synthesized
gccaagattgtgccatttcaccccagcctggcaacagagcaagacaccatctaaaaaaaaaaaaaaaaaaaaaaaaaattcaatgctgacacaaataaggtttcaattaaacaacttctt

SEQ ID NO: 35 - DNA, 120 bp, Artificial sequence
  The sequence is synthesized
ttttttaaattatctgtttcaggaagaagaacgattatccattcaaaattttaggtaaattttttacttttagtaaaaaatttttttctttttatagaagtaagtattttataatctttt

SEQ ID NO: 36 - DNA, 120 bp, Artificial sequence
  The sequence is synthesized
aaaaaaaaaaaaaattcaatgctgacacaaataaggtttcaattaaacaacttctttttttttttttaaattatctgtttcaggaagaagaacgattatccattcaaaattttaggtaaa

SEQ ID NO: 37 - DNA, 120 bp, Artificial sequence
  The sequence is synthesized
tgctaataatttgtttaatgaaataggtagttcctaaggtttatatctgttagtaagaggtttatttgaggggaagtgaaagaacttgaaagattcatggtctctaaatttttttttttt

SEQ ID NO: 38 - DNA, 120 bp, Artificial sequence
  The sequence is synthesized
ttttttttttcagagatttggaccaggcaagcatggaagcagtagtttcacttctagctggtctccctctgcagcctgaagaaggagatggtgtggaattgatggaagccaaatcacagt

SEQ ID NO: 39 - DNA, 120 bp, Artificial sequence
  The sequence is synthesized
gtttatttgaggggaagtgaaagaacttgaaagattcatggtctctaaattttttttttttttttttttttttttcagagatttggaccaggcaagcatggaagcagtagtttcacttct

SEQ ID NO: 40 - DNA, 120 bp, Artificial sequence
  The sequence is synthesized
catgccattgcactccatcctgagggacagaacaagactctatctcaggaaaaaaaaaaagtaataaaacagtagcaagaaaacaaaacagtagcaagagcaagaataagcagaagtatt

SEQ ID NO: 41 - DNA, 120 bp, Artificial sequence
  The sequence is synthesized
gacagaacaagactctatctcaggaaaaaaaaaaagtaataaaacagtagcaagaaaacaaaacagtagcaagagcaagaataagcagaagtattaaggtggcatcttccataattatcc

SEQ ID NO: 42 - DNA, 120 bp, Artificial sequence
  The sequence is synthesized
aagtaataaaacagtagcaagaaaacaaaacagtagcaagagcaagaataagcagaagtattaaggtggcatcttccataattatcctcaaggaacagggtctacctccatgtttccagt

SEQ ID NO: 43 - DNA, 120 bp, Artificial sequence
  The sequence is synthesized
aggcaacagaacaaaactccatctcaaaaaaaaaaaaaaaaaaaaagatttgtctctgaaattaggttgaaaatatttttattggtttcatcaggcaagaaacttgggtttttagagaag

SEQ ID NO: 44 - DNA, 120 bp, Artificial sequence
  The sequence is synthesized
ctcaaaaaaaaaaaaaaaaaaaaagatttgtctctgaaattaggttgaaaatatttttattggtttcatcaggcaagaaacttgggtttttagagaaggaaaactatggtaggaaactgg

SEQ ID NO: 45 - DNA, 120 bp, Artificial sequence
  The sequence is synthesized
aaaagatttgtctctgaaattaggttgaaaatatttttattggtttcatcaggcaagaaacttgggtttttagagaaggaaaactatggtaggaaactggtgaccagctcttcttttttc

SEQ ID NO: 46 - DNA, 120 bp, Artificial sequence
  The sequence is synthesized
tgtatgcaggtactgaatgacttggcagcctgaggatgaacactttgtaatgagaatctatatttgtggtggatcacaccttcctgaaaacttaagcatttttttcccccattgtttttt

SEQ ID NO: 47 - DNA, 120 bp, Artificial sequence
  The sequence is synthesized
ttttttttatagagaaagctaggaacattgccattttcctcatagtatctcatgattaaataatgacaagggagaataaactaaaatgtgtttttcccctaagttgctgcaaagattaaa

SEQ ID NO: 48 - DNA, 120 bp, Artificial sequence
  The sequence is synthesized
ttgtggtggatcacaccttcctgaaaacttaagcatttttttcccccattgttttttttttttttatagagaaagctaggaacattgccattttcctcatagtatctcatgattaaataa

SEQ ID NO: 49 - DNA, 120 bp, Artificial sequence
  The sequence is synthesized
ctttcttctcaagcttaatgatacagtcaagtgaatttattttctaaactatgatgacctcttattcatgatagtagctattcccactataagggagaaaggattatcttctcaaaaaaa

SEQ ID NO: 50 - DNA, 120 bp, Artificial sequence
  The sequence is synthesized
aaaaaacacagaataaaagcacactgtataaaattacctggaggagaagtgacagtaaaaataaagtgccagtttgacccagagatcaagactgccaagttgtagaagctaacataacca

SEQ ID NO: 51 - DNA, 120 bp, Artificial sequence
  The sequence is synthesized
tcatgatagtagctattcccactataagggagaaaggattatcttctcaaaaaaaaaaaaaaaaaacacagaataaaagcacactgtataaaattacctggaggagaagtgacagtaaaa

SEQ ID NO: 52 - DNA, 120 bp, Artificial sequence
  The sequence is synthesized
gatatgcaagagctgtactaagctgataggcatacaggatcaaagatgctagatccaaactgtatttccttacttgcaaaaatgacctcagcttttggaacaatgaccaaaagaaaaaaa

SEQ ID NO: 53 - DNA, 120 bp, Artificial sequence
  The sequence is synthesized
aaagaattagtggcagtgaatcgaaatcaaaagcaaaaaagctacattcaacaataatgctgttttcaatgcatttttaataatattagaatggcttcaagtctcttctaataaagcata

SEQ ID NO: 54 - DNA, 120 bp, Artificial sequence
  The sequence is synthesized
tgtatttccttacttgcaaaaatgacctcagcttttggaacaatgaccaaaagaaaaaaaaaaaaaaaagaattagtggcagtgaatcgaaatcaaaagcaaaaaagctacattcaacaa

SEQ ID NO: 55 - DNA, 120 bp, Artificial sequence
  The sequence is synthesized
tttaaagcagttcttttctagcccgtgtgtaactatacaaataattgagtgttcatgttctcaggaacagagatgaaaaactcctgctaagatcggaattttaaattaatgatttttttt

SEQ ID NO: 56 - DNA, 120 bp, Artificial sequence
  The sequence is synthesized
tttttgtccttgaagaagccttattctcaccatccctcactcacttccctacttcccacagtggcattgggcctctacttctcgagggatgcttactgggagaagctgtatgtggaccag

SEQ ID NO: 57 - DNA, 120 bp, Artificial sequence
  The sequence is synthesized
ctcaggaacagagatgaaaaactcctgctaagatcggaattttaaattaatgattttttttttttttgtccttgaagaagccttattctcaccatccctcactcacttccctacttccca

SEQ ID NO: 58 - DNA, 120 bp, Artificial sequence
  The sequence is synthesized
tgacctgcaatgattatatttcggtttttgaatttgacatctttacccgactctttcaggtaggacactaaaaaagttgactaaactggttactgctacttcggtgaagagaaagctttt

SEQ ID NO: 59 - DNA, 120 bp, Artificial sequence
  The sequence is synthesized
ttttaaataacatttggggtttttgtctgtatgaaagtattttaattcattttaggaactatgccaaaaaaaagattaaaactatctcatattaaaaatatatgtatgtgtgtgtatgta

SEQ ID NO: 60 - DNA, 120 bp, Artificial sequence
  The sequence is synthesized
ctaaaaaagttgactaaactggttactgctacttcggtgaagagaaagctttttttttttttttttaaataacatttggggtttttgtctgtatgaaagtattttaattcattttaggaa

SEQ ID NO: 61 - DNA, 120 bp, Artificial sequence
  The sequence is synthesized
atacattttctggctgaacagaaaacacggtgaaacgatttgaaaacttttattgtgaattacagggtcctatgaaccctctgtccgtgcctttatgaatatcaacatagacatgttttt

SEQ ID NO: 62 - DNA, 120 bp, Artificial sequence
  The sequence is synthesized
ttttttttgcattaacaccgttttctgtaatattttctttattttacatcaactgctgtactcgatcagccccttaacacgactcgtataaatgctgctgaaatagaaagcagagttcga

SEQ ID NO: 63 - DNA, 120 bp, Artificial sequence
  The sequence is synthesized
cagggtcctatgaaccctctgtccgtgcctttatgaatatcaacatagacatgtttttttttttttttttgcattaacaccgttttctgtaatattttctttattttacatcaactgctg

SEQ ID NO: 64 - DNA, 120 bp, Artificial sequence
  The sequence is synthesized
cagcgccataatcggcaatatataacatgtggcaatccttttcgatgggatacctggagacgaccatcaagagacctgttgggaagcaaggggaaaagaaaggagggaactttaaaaaaa

SEQ ID NO: 65 - DNA, 120 bp, Artificial sequence
  The sequence is synthesized
aaaaaagtgcagtcaaatgggctaaattagtacaataaaagcataaagaaaagaatgacagccatttagttagttttagtgagaatcaagaacaaaaatgtactatttaatggatctgat

SEQ ID NO: 66 - DNA, 120 bp, Artificial sequence
  The sequence is synthesized
cgaccatcaagagacctgttgggaagcaaggggaaaagaaaggagggaactttaaaaaaaaaaaaaaaaaagtgcagtcaaatgggctaaattagtacaataaaagcataaagaaaagaa

SEQ ID NO: 67 - DNA, 120 bp, Artificial sequence
  The sequence is synthesized
ttctcaagcttaatgatacagtcaagtgaatttattttctaaactatgatgacctcttattcatgatagtagctattcccactataagggagaaaggattatcttctcaaaaaaaaaaaa

SEQ ID NO: 68 - DNA, 120 bp, Artificial sequence
  The sequence is synthesized
aaaaaaaaacacagaataaaagcacactgtataaaattacctggaggagaagtgacagtaaaaataaagtgccagtttgacccagagatcaagactgccaagttgtagaagctaacataa

SEQ ID NO: 69 - DNA, 120 bp, Artificial sequence
  The sequence is synthesized
atagtagctattcccactataagggagaaaggattatcttctcaaaaaaaaaaaaaaaaaacacagaataaaagcacactgtataaaattacctggaggagaagtgacagtaaaaataaa

SEQ ID NO: 70 - DNA, 120 bp, Artificial sequence
  The sequence is synthesized
agctttcttctcaagcttaatgatacagtcaagtgaatccattttctaaactatgatgacctcttattcatgatagtagctattcccactataagggagaaaggattatcttctcaaaaa

SEQ ID NO: 71 - DNA, 120 bp, Artificial sequence
  The sequence is synthesized
aaaacacagaataaaagcacactgtataaaattacctggaggagaagtgacagtaaaaataaagtgccagtttgacccagagatcaagactgccaagttgtagaagctaacataaccatg

SEQ ID NO: 72 - DNA, 120 bp, Artificial sequence
  The sequence is synthesized
tattcatgatagtagctattcccactataagctagaaaggattatcttctcaaaaaaaaaaaaaaaaaacacagaataaaagcacactgtataaaattacctggaggagaagtgacagta

SEQ ID NO: 73 - DNA, 120 bp, Artificial sequence
  The sequence is synthesized
tttcttctcaagcttaatgatacagtcaagtgaatttattttctaaactatgatgacctcttattcatgatagtagctattcccactataagggagaaaggattatcttctcaaaaaaaa

SEQ ID NO: 74 - DNA, 120 bp, Artificial sequence
  The sequence is synthesized
aaaaacacagaataaaagcacactgtataaaattacctggaggagaagtgacagtaaaaataaagtgccagtttgacccagagatcaagactgccaagttgtagaagctaacataaccag

SEQ ID NO: 75 - DNA, 120 bp, Artificial sequence
  The sequence is synthesized
tcatgatagtagctattccctctataagggagaaaggattatcttctcaaaaaaaaaaaaaaaaaacacagaataaaagcacactgtataaaattacctggaggagaagtgacagtaaaa